(19) United States
(12) Patent Application Publication
(10) Pub. No.: US 2013/0184645 A1
(43) Pub. Date: Jul. 18, 2013

(54) CATHETER ASSEMBLY WITH IMPROVED SAFETY MEANS

(71) Applicant: Rishi Baid, New Delhi (IN)

(72) Inventor: Rishi Baid, New Delhi (IN)

(21) Appl. No.: 13/825,433

(22) PCT Filed: Sep. 21, 2011
(86) PCT No.: PCT/IB2011/054137

§ 371 Date: Mar. 21, 2013

(30) Foreign Application Priority Data
Sep. 21, 2010 (IN) 2253/DEL/2010

(51) Int. Cl. A61M 25/06

(52) U.S. Cl. CPC A61M 25/0618
USPC 604/164.08

(57) ABSTRACT

A catheter assembly including a catheter; a catheter hub having a distal section and a proximal section, wherein the distal section is joined to the catheter and the proximal section defines a chamber; a needle extending through the catheter hub and the catheter and defining an axial direction, wherein the needle has opposite proximal and distal ends, the distal end forming a needle tip; a needle hub attached to the proximal end of the needle; and a needle guard slidably arranged on the needle, wherein the needle guard is retained in the chamber of the catheter hub when the needle extends through the catheter hub and the catheter, and wherein the needle guard is removable from the catheter hub once the needle tip is received in the needle guard upon withdrawal of the needle from the catheter.

### CROSS-REFERENCE TO THE RELATED APPLICATION

This application claims priority from Indian Patent Application No. 2253/DEL/2010, filed on Sep. 21, 2010, the entire disclosure of which is incorporated herein by reference.

### FIELD OF THE INVENTION

The present invention relates in general to the intravenous catheter assembly. More particularly, the present invention relates to a catheter assembly with improved safety means that automatically covers the sharp tip of the needle after withdrawal of the needle from the catheter and catheter hub.

### BACKGROUND OF THE INVENTION

A catheter assembly of the above-kind is generally known and typically used to facilitate insertion and placement of a catheter or another medical device into a patient's vasculature.

Due to the increasing incidence of blood-borne pathogens such as human immunodeficiency virus (HIV), hepatitis B virus (HBV) and hepatitis C virus (HCV) there is a constant need to protect clinicians or other healthcare workers or personnel handling catheter assemblies from accidental contact with the sharp needle tip after withdrawal of the needle from the catheter and catheter hub.

### SUMMARY OF THE INVENTION

The invention in its broadest aspect embodies an intravenous catheter assembly comprising a catheter, a catheter hub having a distal section and a proximal section, wherein the distal section is joined to the catheter and the proximal section defines a chamber, a needle extending through the catheter hub and the catheter and defining an axial direction, wherein the needle has opposite proximal and distal ends, the distal end forming a tip, wherein the distal end of the said needle has a different dimension (bulge or crimp) proximal to the said needle tip, a needle hub attached to the proximal end of the needle; and a needle guard slidably arranged on the needle for protecting the said needle tip having a base portion having a bore extending in an axial direction through the bore for receiving a needle; and two opposing jaws extending from the base portion generally in the axial direction and each having a head portion in the region of its free end characterized in that the said needle guard is secured partly or completely in the chamber of the catheter hub or completely outside the catheter hub by means configured inside and/or outside of the said needle guard and by means configured inside and/or outside of the said catheter hub when the needle extends through the said catheter hub and the catheter, wherein the said needle guard is removable from the catheter hub once the slot or crimp proximal to the said needle tip is received in the needle guard upon withdrawal of the needle from the said catheter and wherein the said needle guard comprising a resilient member or elastic element or tension ring surrounding the two opposing jaws integrated therewith in a region between the base portion and the head portions of the said needle guard.

The needle as described has a bulge or crimp proximal to the tip of the needle. This bulge or crimp is used as an obstruction when the needle guard is in the protective position i.e. covering the needle tip. As the needle guard slides along the needle the bulge or crimp proximal to the needle tip engages with the base of the needle guard and locks the needle guard over the tip, thereby preventing slipping.

The means configured on the inner surface of the needle guard may comprise at least one engaging and/or retaining projection and/or recess and/or a combination of a projection and a recess capable of securing needle guard partially and/or completely in the chamber of the catheter hub.

The means configured on the outer surface of the needle guard may comprise retaining and/or engaging arms connected to the base portion of the needle guard retaining and/or engaging partially and/or completely in the chamber of the catheter hub when the needle extends through the catheter hub and the catheter, and wherein the needle guard is prompted to protect the needle tip once the bulge, hole, slot and/or depression proximal to the said needle tip is received in the needle guard upon withdrawal of the needle from the catheter.

The means configured on the inner surface of the catheter hub may comprise at least one annular projection and/or recess and/or a combination of a projection and a recess configured inside the said catheter hub to secure the needle guard partially and/or completely in the chamber of the catheter hub.

The means configured on the outer surface of the catheter hub may comprise at least one engaging and/or retaining projection and/or recess and/or a combination of a projection and a recess to engage and/or retain the needle guard partially and/or completely in the chamber of the catheter hub.

It is thus a primary object of this invention to provide a catheter assembly which can be manufactured at a low cost and which is easy in its operation and design also same provides reliable protection against accidental pricking by the needle once the needle has been withdrawn from the catheter and catheter hub.

### BRIEF DESCRIPTION OF THE DRAWINGS

FIGS. 1A and B show side views of a catheter assembly of the invention prior to use;

FIG. 2 shows a perspective view of a needle, a needle hub attached thereto and a needle guard guarding a tip of the needle after use of the catheter assembly of FIG. 1;

FIGS. 3A to C show perspective views (A and B) and a side view (C) of the needle guard of FIG. 2; and

FIG. 4 shows a longitudinal sectional view of a needle guard received in a catheter hub of the catheter assembly of FIG. 1;

FIGS. 5A and B show a side view (A) and a perspective view (C) of an alternative needle guard; and

FIGS. 6A to C show a side view (A), a perspective view (B) and a longitudinal sectional view (C) of the needle guard of FIG. 5 without tension element.

FIG. 7 shows a longitudinal sectional view of a needle guard partly received in a catheter hub of the catheter assembly of FIG. 1;

FIG. 8 shows a longitudinal sectional view of a needle guard completely outside in a catheter hub of the catheter assembly of FIG. 1;

### DETAILED DESCRIPTION OF THE INVENTION AND DRAWINGS

A reading of all figures together describes the invention embodying an intravenous catheter assembly 10 comprising a catheter 12, a catheter hub 14 having a distal section and a proximal section, wherein the distal section is joined to the catheter 12 and the proximal section defines a chamber 24, a needle 16 extending through the catheter hub 14 and the catheter 12 and defining an axial direction, wherein the needle 16 has opposite proximal and distal ends, the distal end forming a tip 18, wherein the distal end of the said needle has a different dimension (crimp or bulge) proximal to the said needle tip 18, a needle hub 20 attached to the proximal end of the needle 16; and a needle guard 26 slidably arranged on the needle 16 for covering the said needle tip 18 having a base portion 28 having a bore 52 extending in an axial direction through the bore 52 for receiving a needle 16; and two opposing jaws (30, 32) extending from the base portion 28 generally in the axial direction and each having a head portion in the region of its free end wherein the said needle guard 26 is secured partly or completely in the chamber of the catheter hub 14 or completely outside the catheter hub by means 52 configured inside and/or outside of the said needle guard 26 and by means 80 configured inside and/or outside of the said catheter hub 14 when the needle 16 extends through the said catheter hub 14 and the catheter 12, wherein the said needle guard 26 is removable from the catheter hub 14 once the crimp or bulge proximal to the said needle tip 18 is received in the needle guard 26 upon withdrawal of the needle 16 from the said catheter 12 and wherein the said needle guard 26 comprising a resilient member 48 or elastic element 48 or tension ring 48 surrounding the two opposing jaws (30, 32) integrated therewith in a region between the base portion and the head portions of the said needle guard.

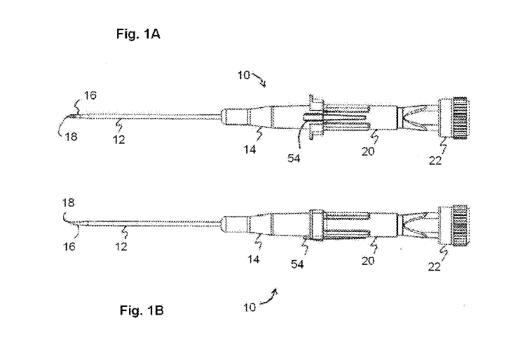

FIG. 1 shows a catheter assembly 10 in accordance with the invention prior to use. The catheter assembly 10 includes a tube-like catheter 12 having distal and proximal ends, and a catheter hub 14 attached to the catheter 12 at the proximal end of the catheter 12.

The catheter assembly 10 further comprises a needle 16 extending through the catheter 12 and catheter hub 14 and defining an axial direction. The needle 16 has distal and proximal ends, wherein a sharp needle tip 18 is formed at the distal end of the needle 16, which protrudes from the catheter 12 at the distal end thereof.

A needle hub 20 is attached to the needle 16 at the proximal end of the needle 16. A port member 22 is mounted to the needle hub 20 at a proximal side of the needle hub 20, which makes it possible to connect, for example, a PVC tube to the catheter assembly 10 for collecting blood from a patient or delivering a fluid to be administered to a patient.

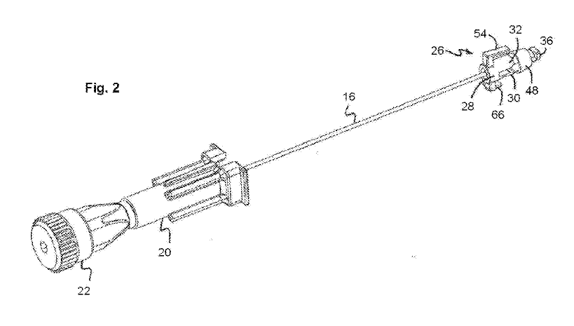

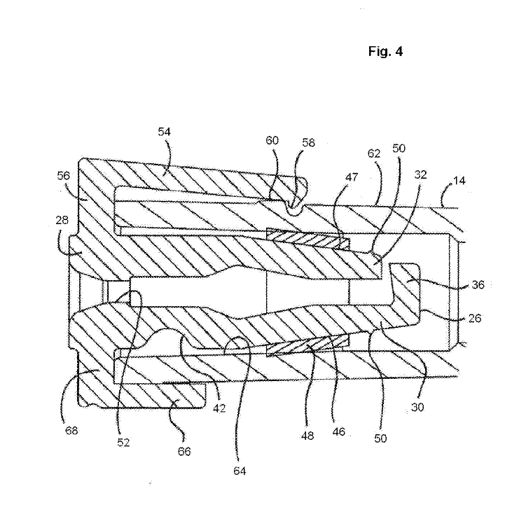

The catheter hub 14 defines an inner chamber 24 (FIGS. 4 & 7) in which a needle guard 26 is fully or partly seated. In FIG. 1 only a retaining arm 54 of the needle guard 26 can be seen. The needle guard 26 is slidably arranged on the needle 16, such that it moves along the needle 16 when the needle 16 is withdrawn from the catheter 12. The purpose of the needle guard 26 is to cover the needle tip 18 after the needle 16 has been used in order to prevent accidental pricking by the needle 16 of a person handling the catheter assembly 10, as can be seen from FIG. 2.

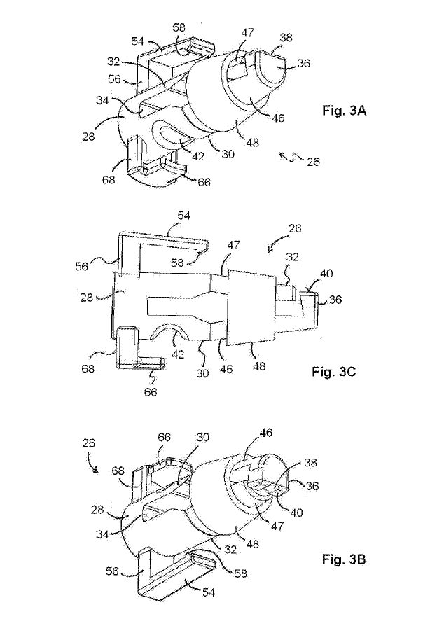

As shown in FIG. 3, the needle guard 26 includes a generally cylindrical base portion 28 at a proximal end of the needle guard 26. First and second arms 30, 32 extend from a distal face 34 of the base portion 28 generally in the axial direction. The first arm 30 is longer than the second arm 32.

A transverse wall 36 is arranged in a distal region of the first arm 30. The dimension of the transverse wall 36 as seen in a radial direction is selected such that the transverse wall 36 overlaps with the second arm 32 when the needle tip 18 is received in the needle guard 26, i.e. between the first and second arms 30, 32, such that the needle tip 18 is blocked by the transverse wall 36 from distally protruding from the needle guard 26 (FIG. 2).

Prior to use, i.e. when the needle 16 extends all the way through the needle guard 26, the transverse wall 36 is supported on the needle 16 and the first arm 30 is deflected away from the second arm 32. An axial groove 38 is provided in the transverse wall 36 on its side 40 facing the needle 16 in order to guide the transverse wall 36 on the needle 16, in particular when the needle 16 is pulled through the needle guard 26 upon withdrawal of the needle 16 from the catheter 12.

A recess 42 is provided in the outer surface of a proximal section 44 of the first arm 30 in order to increase the deflectability of the first arm 30.

Distal sections 46, 47 of the first and second arms 30, 32 have outer surfaces that are tapered towards the distal ends of the first and second arms 30, 32. The first and second arms 30, 32 are surrounded by an elastic band 48 in the region of the distal sections 46, 47. Because of the tapered outer surfaces of the distal sections 46, 47 the elastic band 48 is prevented from sliding off the distal sections 46, 47 towards the proximal end of the needle guard 26 when the first and second arms 30, 32 are spread apart against a restoring force of the elastic band 48 by the needle 16 extending all the way through the needle guard 26. In order to further define the axial position of the elastic band 48, protrusions 50 extending along the outer periphery of the first and second arms 30, 32 can be provided adjacent the elastic band 48 (FIG. 4). These protrusions 50 are not shown in FIGS. 2 and 3.

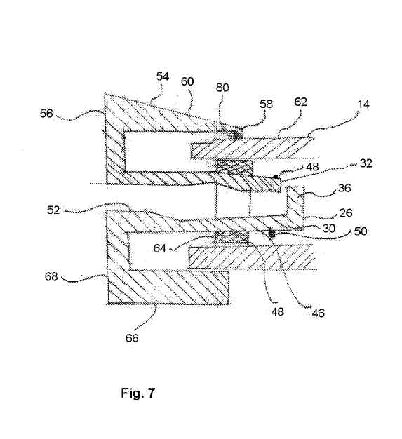

As is best seen in FIGS. 4 and 7, an axial bore 52 extends through the base portion 28 of the needle guard 26 for receiving the needle 16. The profile of the bore 52 is adapted to the principle outer profile of the needle 16. The needle 16 has a change in profile, in particular an enlargement (not shown), near its needle tip 18, which has an outer profile one dimension of which is larger than a maximum dimension of the profile of the bore 52. Thus, the change in profile prevents the needle guard 26 from sliding off the needle 16 when the needle tip 18 is received between the first and second arms 30, 32 and covered by the needle guard 26.

In another construction an axial bore 52 extends through the base portion 28 of the needle guard 26 for receiving the needle 16. The profile of the bore 52 is adapted to the principle outer profile of the needle 16. The needle 16 has a change in profile, in particular a crimp (not shown), near its needle tip 18, which has an outer profile one dimension of which is smaller than a maximum dimension of the profile of the bore 52. Thus, the change in profile prevents the needle guard 26 from sliding off the needle 16 when the needle tip 18 is received between the first and second arms 30, 32 and covered by the needle guard 26.

A retaining arm 54 is provided on the needle guard 26 in order to secure the needle guard 26 in the chamber 24 of the catheter hub and, in particular, to prevent removal of the needle guard 26 from the catheter hub 14 upon withdrawal of the needle 16 before the needle tip 18 has been safely received in the needle guard 26. The retaining arm 54 is connected to the base portion 28 of the needle guard 26 via a transverse segment 56. The transverse segment 56 extends outwardly from the base portion 28 in a generally radial direction at the side of the second arm 32. The retaining arm 54 extends distally from the transverse segment 56 and is slightly tilted towards the second arm 32. Preferably, the retaining arm 45 forms an angle in the range between 0° and 10° with the axial direction, such that a clearing between the retaining arm 54 and the second arm 32 narrows towards a distal end of the retaining arm 54.

A hook-like protrusion 58 is provided in the region of the distal end of the retaining arm 54. The hook-like protrusion 58 of the retaining arm 54 engages behind a corresponding protrusion 60 and into a groove or recess 61 provided at the outer surface 62 of the catheter hub 14. Because of the engagement between the hook-like protrusion 58 of the retaining arm 54 and the combination of protrusion 60 and recess 61 formed on the catheter hub 14, the needle guard 26 is prevented from axial movement relative to the catheter hub 14 and effectively retained at the catheter hub 14 until a pulling force exerted by the needle 16 on the base portion 28 of the needle guard 26 via the change in profile of the needle 16 upon withdrawal of the needle 16 from the catheter 12 becomes great enough to disengage the retaining arm 54 from the protrusion 60 of the catheter hub 14.

It is to be noted that instead of a combination of protrusion 60 and recess 61 formed at the catheter hub 14 it is also possible to provide either a protrusion 60 or a recess 61 in the outer surface 62 of the catheter hub 14 for engagement with the hook-like protrusion 58 of the retaining arm 54.

The retaining of the needle guard 26 in the catheter hub 14 can be further improved if the elastic band 48 engages with an inner surface 64 of the catheter hub 14 in the spread-apart state of the first and second arms 30, 32. Once the needle tip 18 passes the transverse wall 36 and the first and second arms 30, 32 snap together, thereby allowing the elastic band 48 to contract, the elastic band 48 can disengage from the inner surface 64 of the catheter hub 14.

A supporting arm 66 is provided on a side of the needle guard 26 opposite from the retaining arm 54. The supporting arm 66 forms a generally right angle with a transverse segment 68 which extends from the base portion 28 in a generally radial direction. The supporting arm 66 is configured to be in contact with the outer surface 62 of the catheter hub 14 across its substantially entire length and width. The supporting arm 66 thus prevents the needle guard 26 received in the catheter hub 14 from moving in a radial direction within the chamber 24. The supporting arm 66 may also help to secure the needle guard 26 against axial movement with respect to the catheter hub 14, thereby adding to the retaining of the needle guard 26 in the catheter hub 14. However, the supporting arm 66 does not necessarily have to be in such a retaining relationship with the catheter hub 14.

In the embodiment in FIGS. 4 & 7, the retaining arm 54 is provided on the side of the base portion 28 adjacent the second arm 32, and the supporting arm 66 is provided on the side of the base portion 28 adjacent the first arm 30. However, it is generally also possible to provide the retaining arm 54 on the side of the base portion 28 adjacent the first arm 30 and the supporting arm 66 on the side of the base portion 28 adjacent the second arm 32.

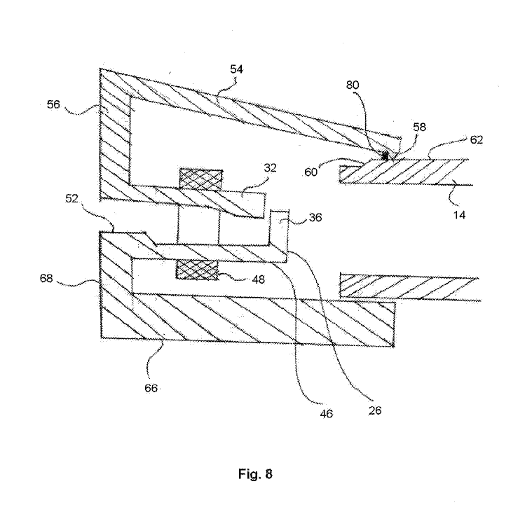

In the embodiments in FIG. 4, the needle guard 26 is completely inside the catheter hub 14 and is retained on the outside by engagement means 80 on the catheter hub 14. The engagement means 80 can be in the form of a groove as shown in FIG. 4 right under hook 58 or in form of a protrusion 80 as shown in FIGS. 7 and 8.

As shown in FIGS. 1 to 4, 7 and 8, the base portion 28, the first and second arms 30, 32, the retaining arm 54 and the supporting arm 66 are integrally formed, for example, from a plastic material by way of injection molding. However, it is to be understood that this integral design is not compulsory. Instead, one or more of the named components could be made of a material that is different from the material of the other components, and this component could be attached to the other components, for example, by gluing, welding, soldering or the like.

Although not shown in FIGS. 1 to 4, 7 and 8, a stopping element may be provided in the base portion 28 or on the distal face 34 of the base portion 28, wherein the stopping element has a through-bore which is aligned with the axial bore 52 in the base portion 28 and which has a profile that is adapted to the principle outer profile of the needle 16. Preferably, such a stopping element would be made of a material of a greater hardness and/or stiffness than the material of the base portion 28, such that this stopping element withstands greater forces exerted by the change in profile of the needle 16 upon withdrawal of the needle 16 from the catheter 12, thereby more effectively preventing the change in profile of the needle 16 from passing through the base portion 28 and thus more effectively preventing the needle guard 26 from sliding off the needle 16.

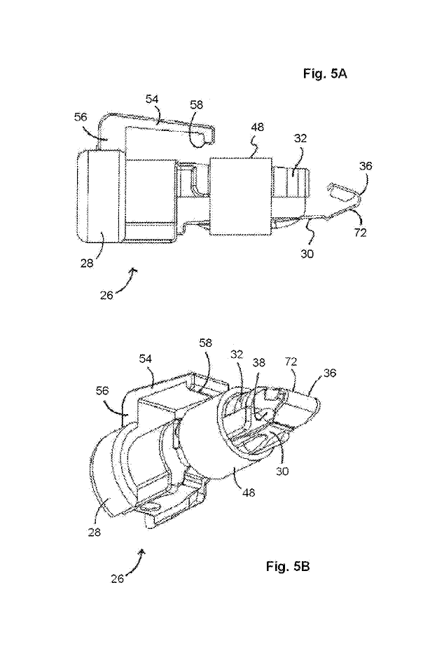

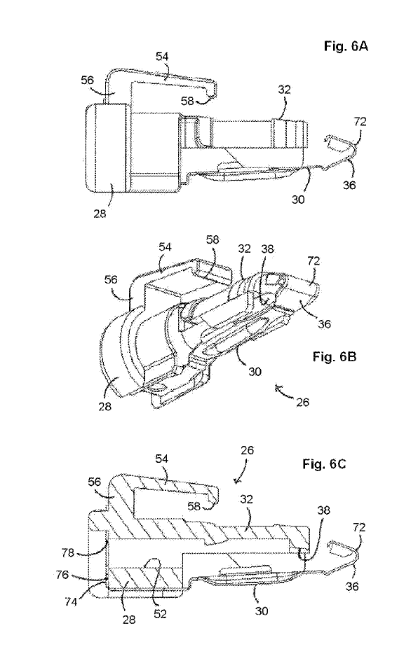

FIGS. 5 and 6 show an alternative needle guard 26 that can be used in the catheter assembly 10 of the invention. This needle guard 26 is generally identical in function but differs from the afore-described needle guard 26 mainly in that it does not have any supporting arm and in that the first arm 30 is not integrally formed with the base portion 28, the second arm 32 and the retaining arm 54.

Instead, the first arm 30 is made of a strip of sheet metal 70, a distal end portion 72 of which is bent to form the transverse wall 36 and a proximal end portion 74 of which is attached, e.g. glued or welded, to a proximal face 76 of the base portion 28. As described above, distal sections 46, 47 of the first and second arms 30, 32 are surrounded by an elastic band 48 such that the first and second arms 30, 32 can be spread apart against a restoring force of the elastic band 48 (FIG. 5).

As is best seen in FIG. 6C, a through-bore 78 is provided in the proximal end portion 74 of the strip of sheet metal 70 and aligned with the axial bore 52 in the base portion 28. The profile of the through-bore 78 is adapted to the principal outer profile of the needle 16, whereas the profile of the axial bore 52 is somewhat wider. Hence, in this needle guard 26 the change of profile of the needle 16, e.g. its enlargement, can slide along the axial bore 52 and is limited in movement in the proximal direction only upon engagement with the proximal end portion 74 of the strip of sheet metal 70 at the proximal side of the base portion 28. The proximal end portion 74 of the strip of sheet metal 70 thus functions as a stopping element of the kind that has already been discussed above and helps to prevent the needle guard 26 from sliding off the needle 16.

In further contrast to the needle guard 26 shown in FIGS. 1 to 4, 7 and 8, in the needle guard 26 of FIGS. 5 and 6 the axial groove 38 for guiding the needle 16 upon movement relative to the needle guard 26 is not provided on the first arm 30 but on the second arm 32, as is best seen in FIG. 6B.

FIGS. 4, 7 and 8 are embodiments of the above described invention and function substantially in the same manner and for the sake of brevity repetition is being avoided.

As can be seen from the description above that base portion 28, one of first arm 30 of the said needle guard are integrally made from different material, e.g. a plastic material, and the second arm 32 is made from a second material different, such as steel, from said first material. It is well within the scope of this invention that the base portion 28, first arm 30, second arm 32 and retaining arm 54 can be made of same material. Likewise, it is well within the scope of the invention to have all the elements of needle guard as described to be unitary or in separate parts and/or different materials.

### REFERENCE NUMERAL LIST

10 catheter assembly
12 catheter
14 catheter hub
16 needle
18 needle tip
20 needle hub
22 port member
24 chamber
26 needle guard
28 base portion
30 first arm
32 second arm
34 distal face
36 transverse wall
38 axial groove
40 side
42 recess
44 proximal portion
46 distal section
47 distal section
48 elastic band
50 protrusion
52 axial bore
54 retaining arm
56 transverse segment
58 protrusion
60 protrusion
61 recess
62 outer surface
64 inner surface
66 supporting arm
68 transverse segment
70 strip of sheet metal
72 distal end portion
74 proximal end portion
76 proximal face
77 through-bore
80 Protrusion or groove on the outside of the catheter hub

1. A catheter assembly comprising:
a catheter;
a catheter hub having a distal section and a proximal section, wherein the distal section is joined to the catheter and the proximal section defines a chamber;
a needle extending through the catheter hub and the catheter and defining an axial direction, wherein the needle has opposite proximal and distal ends, the distal end forming a needle tip;
a needle hub attached to the proximal end of the needle; and
a needle guard slidably arranged on the needle, wherein the needle guard is partly or completely retained in the chamber of the catheter hub or the needle guard is completely outside the catheter hub by means of a retaining arm engaging the outside of the catheter hub when the needle extends through the catheter hub and the catheter, and wherein the needle guard is removable from the catheter hub once the needle tip is received in the needle guard upon withdrawal of the needle from the catheter.

2. A catheter assembly in accordance with claim 1, wherein a proximal end of the retaining arm is connected to a base portion of the needle guard by means of a transverse segment, wherein the transverse segment extends generally in a radial direction and/or from a proximal end region of the needle guard.

3. A catheter assembly in accordance with claim 1, wherein the retaining arm extends not parallel to the axial direction and forms an angle in the range between 0° and 10° with the axial direction, such that a clearing between the retaining arm and the catheter hub narrows towards a distal end of the retaining arm.

4. A catheter assembly in accordance with claim 1, wherein a hook-like protrusion is provided in the region of a distal end of the retaining arm.

5. A catheter assembly in accordance with claim 1, wherein a projection or a recess or a combination of a projection and a recess is provided on the outer surface of the catheter hub for engagement with the retaining arm with a hook-like protrusion provided in the region of a distal end of the retaining arm.

6. A catheter assembly in accordance with claim 1, wherein at least one supporting arm for engaging the outer surface of the catheter hub is provided on a side of the needle guard opposite from the retaining arm.

7. A catheter assembly in accordance with claim 1, wherein the needle guard comprises a generally cylindrical base portion in the region of its proximal end and first and second arms extending in a generally axial direction from a distal side of the base portion, wherein the first and second arms are surrounded by a tension element in a distal region of the first and second arms.

8. A catheter assembly in accordance with claim 7, wherein the first and second arms are spread apart by the needle extending completely through the needle guard such that the tension element is brought into retaining engagement with an inner surface of the catheter hub.

9. A catheter assembly in accordance with claim 7, wherein a transverse wall is arranged in a distal region of one of the first and second arms, wherein a groove is provided in a side of the transverse wall, the groove extending substantially in the axial direction.

10. A catheter assembly in accordance with claim 1, wherein the base portion of the needle guard has an axial bore extending therethrough for receiving the needle, wherein the profile of the bore is adapted to the principal outer profile of the needle, and the needle has a change in profile near the needle tip, said change in profile having an outer profile one dimension of which is larger than a maximum dimension of the profile of the bore.

11. A catheter assembly comprising:
a catheter;
a catheter hub having a distal section and a proximal section, wherein the distal section is joined to the catheter and the proximal section defines a chamber;
a needle extending through the catheter hub and the catheter and defining an axial direction, wherein the needle has opposite proximal and distal ends, the distal end forming a needle tip;
a needle hub attached to the proximal end of the needle; and
a needle guard slidably arranged on the needle, wherein the needle guard is partly or completely retained in the chamber of the catheter hub or the needle guard is completely outside the catheter hub by means of a retaining arm engaging the outside of the catheter hub when the needle extends through the catheter hub and the catheter, and wherein the needle guard is removable from the catheter hub once the needle tip is received in the needle guard upon withdrawal of the needle from the catheter, wherein
a proximal end of the retaining arm is connected to a base portion of the needle guard by means of a transverse segment, and wherein
the transverse segment extends generally in a radial direction and/or from a proximal end region of the needle guard, and wherein
wherein the retaining arm extends not parallel to the axial direction and, in particular, forms an angle in the range between 0° and 10° with the axial direction, in particular such that a clearing between the retaining arm and the catheter hub narrows towards a distal end of the retaining arm, and further wherein
a hook-like protrusion is provided in the region of a distal end of the retaining arm.

12. A catheter assembly in accordance with claim 11, wherein a projection or a recess or a combination of a projection and a recess is provided on the outer surface of the catheter hub for engagement with the retaining arm with a hook-like protrusion provided in the region of a distal end of the retaining arm.

13. A catheter assembly in accordance with claim 11, wherein at least one supporting arm for engaging the outer surface of the catheter hub is provided on a side of the needle guard opposite from the retaining arm.

14. A catheter assembly in accordance with claim 11, wherein the needle guard comprises a generally cylindrical base portion in the region of its proximal end and first and second arms extending in a generally axial direction from a distal side of the base portion, wherein the first and second arms are surrounded by a tension element in a distal region of the first and second arms.

15. A catheter assembly in accordance with claim 14, wherein the first and second arms are spread apart by the needle extending completely through the needle guard such that the tension element is brought into retaining engagement with an inner surface of the catheter hub.

16. A catheter assembly in accordance with claim 14, wherein a transverse wall is arranged in a distal region of one of the first and second arms, wherein a groove is provided in a side of the transverse wall, the groove extending substantially in the axial direction.

17. A catheter assembly in accordance with claim 14, wherein the base portion of the needle guard has an axial bore extending therethrough for receiving the needle, wherein the profile of the bore is adapted to the principal outer profile of the needle, and the needle has a change in profile near the needle tip, said change in profile having an outer profile one dimension of which is larger than a maximum dimension of the profile of the bore.

18. A catheter assembly in accordance with claim 11, wherein the base portion of the needle guard has an axial bore extending therethrough for receiving the needle, wherein the profile of the bore is adapted to the principal outer profile of the needle, and the needle has a change in profile near the needle tip, said change in profile having an outer profile one dimension of which is larger than a maximum dimension of the profile of the bore.
